SEQUENCE LISTING (2 sequences)

SEQ ID NO: 1 - DNA, 1491 bp, Apis mellifera sinisxinyuan
atgccaacgaaccacccaccagatcgcttgttcgctatgtcgcgaactccctcttactttctctctctcacgttaggacgtttgatcagtttatctcgctcgcttctccattatccttctctatccgactcatatcgttgcttgtcatcttatcttcgtccctctcgaatgcgcggaggcagacgccaacgaattcacttcacgcaacttcagttttactcggtcagttcctactgccggccagaagaattcgtcagtattcgccgcgctgcattgccgaactctataaggcacaacttatcgttacatagcagatttatgcgagtgcagaacgagggtaccggaaagagttcctggtggatgatcaatcgtgatgcgaaaccagggaaatcacgtagaagagcaataaccatggagacgagcaaatttgagaaacgacgtggtagggtaaggaagaagatcgaggcgctaaaaaacggtgggttacaagctgacacgacaactagccccagtaacagtgtgaatgaggggttggatctctttccagacagtccacttcagtctggcagtggatttcaactttcacctgattttcgtcctcgagcttcgagcaatgcctcatcctgtggtcgattaagtccaatcacggcgattcctaggaaaccggaatggacaccaacatacacactctcatatagtccagaacaattagctggtagcctcgcagaaacgatgaaattggaatcctatcaaatgtatcatacgacacaaccgagccaccagcatcatacaggaccacctccttcttattacgaatcccaatatcagaggagcaatagcttgtcgtcaggatcgtcctccttcgctctgcagccaactcctcaatcagcaaatcaacaaagatgcccaattcacggtttgcaaccgtgtgcctgtcaaatgaatttgagtccagtggctggaatgtcgccatcgtaccagcaaagcgaaccaagtcccacaacgttaggaaataatcaacagcaaactcttcaatacatgatgcagtcgcaacaatgtcagcagcaacaattgccacaacgacaacagcaatcacaaacaggagtaacagattccagttcatcacaaacatctaccccatgtgaccccacaccaagcactatgatgggtcaattgatgggagccttgaacaattcaacacttcttgatgatttaaatattaacatcgaatcgctgcatggtggatttgattgtaacgtagaagaggtgatcaaacacgagttgtcgatggatggtacattagatttcaacttccaacagagtgtaatgggtacggctgcaatccaagtaagcgacaataatatgggtcagaacggcgctgtttcgcagaataacgtgataggtacgacgacgaccggaaacgcgaccgcaggtgtttacgtgagcaccaatgcggccactcctgctgcacctccctcttgggttcattag

SEQ ID NO: 2 - DNA, 2583 bp, Apis mellifera sinisxinyuan
atgggcactatacttcaacaatcggtgttaaaaggccgacaaagaattaataatgaaaaagaaaaaaatttacttcatgaattattcactcagacagcagtaaattattccaatcaagtggcaatctattataaagatgacaatggccaggaatatgtaatttcttatgaacaattaaacgagataaccaataaattggccagagtttttcaaaaacttgagaaatcagaagatgcatcaaaatcatttgtagcggtatgcatgaaaccctcgcatcaattaccgaccgtactgattgccatattgaaggctggtatggcatatctgcccttggacgcagaattccctatggtcaggatgaaacatatcttagaagaagcaaaaccttttgtggtggtaatcgaacaagaaacggatttatcactttacaaaggaacaaacacgatcacctatgaacaattgttaaagcaatctaaaaatgaagagaaagaaaatttgaaggttgaagaagatcctgaaagaaccgcaattgttttatatacttctggaagcacaggcatacctaaaggagttcttctaccacatgcaactatattgaatcgattggaatggcaatggcaggaattaccttatgcagaggacgaaaaaaattgtatttttaagacatctttgacatttgtggatagtgtatctgaaatttggggacctttgttgcaaggacgtacactggtggttgttcccaaacatgttaccaaggatccagagagatttgtgtctgtactggaagaacataagattcagcgattggtactggtcccatccttattgcactccatgttaatgtatctcagtttacaaaacaaggatgatgttctacgttcgttaagactttgggtatgctctggagagacattgccagtttctttagcggatcaattcttcaccacatttgagaatgacagtaaaattttagcaaatttttatggaagtacagagatcatgggagatgttacctattatcttttgaccaatcggaagcaactacagtcggtagaaaaagtaccaattggaaaaccactggacaattgcgttatttatatcgtcaacaaagatatgcgactaataccacaaggagaagttggagaattaatagttgccggtagaaaccttgcaacaggctatattcacaataatgaatcccgcaagtttcaagacaatccttatacgatcgatttagaatattcaaggatttattgcactggagattatgccaaaatatcaaaaggtgtgattatatacgaaggacgtgtagattcgcaaattaaagttaggggacatcgtgtggatcttacggaagtagaaaaagtaatatctaggatatctgaaatagatggagtggttgttctttgttacaaacccggtgaattgtcacagagtctagttgcttttgtaactattgcaaaagattcgtccacttgcgtatcaaaaatagaaacgtttctacaatctatgctacctatatacatgttaccacgagttatcatcgtggaaaatataccacttttaacaaatgggaagactgatcgtcaggcattattaaaatattatgaatcttctaatgtcaataacgaaaacgagcagtgtatgaattgtgactatactaatgtacttgagaaggatcttgccaaggcgaaagttctgtttccaacagtggcatccgtgattggaaatggaggtcgtgcagacatcacgcttaatgcaaatttttatgaacttggcgggaattctttgaattcgatttatactgtgacgaagctccgggatcaaggttatgaaataggcattacagaatttatcacggcaaaaaatttggccgaggtgctcgatcgaatgaagattagtactgaagacgtggtttatgaaaatactcttaacgaagaagagcatatctttgaaatgttaaatgattctcataaaaaagatgtaatagaaattattacagaaagtttctacagtaaagcagatttagaacagtggttaatgccggatataacaagggaagattatcaaataatgatggaaatgctttggaattctttagtggaaaaaaatttaagttttgtagtgaaatcatcgcaagataataaaacaataggagtaggcttgaattttgatctctgggatgagcctgaattaatccttgattccaaattgatgatcgttttcgaatttttggaatatctagaagcgcctattagagaacgaaaattaccaaaaggaaaaggtcagattattcacaatttcatgatgacaacaagcagtgatttaaatccggccgaaaatgtaattatgatgaaagagatggaagaatattgtttgcaattggctaaaaggaaagaatatgctggcattttcactacaaatacaagtccattaacacagcaacttggaatagacgtgttcggatatgaaacaatgttgacttatcaagtaaataggtattatgctccagatggtacaaaaccttttgaaaaagctccggacgatcaactggcaatttgctcgttgaaaatgatcacttaa

(19) United States
(12) Patent Application Publication
(10) Pub. No.: US 2017/0218461 A1
(43) Pub. Date: Aug. 3, 2017

(54) Gene And Use Thereof

(71) Applicant: INSTITUTE OF APICULTURAL RESEARCH, CHINESE ACADEMY OF AGRICULTURAL SCIENCES, Beijing (CN)

(72) Inventors: Chao CHEN, Beijing (CN); Wei SHI, Beijing (CN); Zhiguang LIU, Beijing (CN); Xiao CHEN, Beijing (CN); Huihua WANG, Beijing (CN); Haikun GUO, Beijing (CN)

(73) Assignee: INSTITUTE OF APICULTURAL RESEARCH, CHINESE ACADEMY OF AGRICULTURAL SCIENCES, Beijing (CN)

(21) Appl. No.: 15/412,746

(22) Filed: Jan. 23, 2017

(30) Foreign Application Priority Data
Jan. 27, 2016 (CN) 201610055405.4

(51) Int. Cl. C12Q 1/68; A01K 67/033; C07K 14/435

(52) U.S. Cl. CPC C12Q 1/6888; C07K 14/43572; A01K 67/033; C12Q 2600/158; C12Q 2600/124; C12Q 2600/156

(57) ABSTRACT

The present invention relates to the field of biotechnology, in particular to genes and use thereof. The present invention employs whole genome sequencing to perform whole genome re-sequencing on a large number of individuals of the honey bee Apis mellifera sinisxinyuan, and obtains genes specific to the A. m. sinisxinyuan. The genes play important roles in the differentiation of A. m. sinisxinyuan from the honey bees in other regions and in the adaptive evolution of A. m. sinisxinyuan to the local environment. The Foxo gene or the Ebony gene provided in the present invention can be used to identify A. m. sinisxinyuan from other subspecies; can also be used for studying the genetic diversity of species resources of bees; and can further be used for studying cold resistance genes. This will fill the gap in the research field of A. m. sinisxinyuan by Chinese researchers.

### CROSS REFERENCE OF RELATED APPLICATION

The present application claims the priority of China Patent Application No. 201610055405.4, filed with the Patent Office of China on Jan. 27, 2016, titled “GENE AND USE THEREOF”, the contents of which are incorporated herein by reference in its entirety.

### FIELD OF THE INVENTION

The present invention relates to the field of biotechnology, in particular to genes and use thereof.

### BACKGROUND OF THE INVENTION

With a long history of apiculture, the germplasm resources of honey bees are rich in China; however, for a long period of time, only eastern honey bees rather than western honey bees have been found in the territory of China. Since the collection ability of the eastern honey bees is slightly inferior to that of the western honey bees, an increasing number of Chinese beekeepers begin to breed the western honey bees with the introduction thereof, and thus the germplasm resources of native bees are threatened in China. The introduced resources become a threat to the local populations; meanwhile problems exist within the introduced resources that they are to some extent inadaptable to the local environment. If native western honey bees can be found in the territory of China, it will be of critical significance to the germplasm resources of honey bees in China. As the director of Chinese Honey Bee Germplasm Resources Committee, Dr. Shi Wei along with the staff of Xinjiang Autonomous Region apiculture management station have been engaged in strengthening the protection work of the original Yili dark bee of Xinjiang, and with several years of efforts, they discovered a new subspecies of the western honey bee (Apis mellifera sinisxinyuan) for the first time in the territory of Yili, Xinjiang of China, which has a differentiation of at least 132,000 years from the other western honey bee subspecies currently known internationally, demonstrating that China is also an origin of western honey bees and terminating the history that there is no natural distribution of western honey bees in China, which is a great breakthrough in the aspects of livestock and poultry resources research. A. m. sinisxinyuan has large body, good performance for wintering, strong stress resistance, outstanding ability of disease resistance and strong oviposition ability of the queen which can maintain a strong group and huge collection ability of the bee colony; A. m. sinisxinyuan can perform both as a new species to be popularized and as a breeding material for further in-depth breeding research, which possesses good development prospects.

The native western honey bee discovered in China has a great impact at home and abroad, however, none of the genes has been recorded to be important for the adaptation of A. m. sinisxinyuan to the local environment. In order to more deeply protect A. m. sinisxinyuan and to effectively employ A. m. sinisxinyuan for services of honey bee breeding in China, it is of great practical significance to provide the genes specific to the A. m. sinisxinyuan.

### SUMMARY OF THE INVENTION

In view of this, the present invention provides genes of Apis mellifera sinisxinyuan and use thereof. The present invention employs whole genome sequencing to perform whole genome re-sequencing on a large number of individuals of A. m. sinisxinyuan and obtains genes specific to A. m. sinisxinyuan.

In order to achieve the above inventive object, the present invention provides the following technical solutions:

The present invention provides a polynucleotide having:

(I) the nucleotide sequence set forth in SEQ ID No. 1 (Foxo gene) or SEQ ID No. 2 (Ebony gene); or

(II) a sequence complementary to the nucleotide sequence set forth in SEQ ID No. 1 or SEQ ID No. 2; or

(III) a sequence which encodes the same protein as that the nucleotide sequence of (I) or (II) does but differs from the nucleotide sequence of (I) or (II) due to genetic codon degeneracy; or

(IV) a nucleotide sequence having a nucleotide sequence obtained from the nucleotide sequence set forth in SEQ ID NO: 1 or SEQ ID No. 2 by substitution, deletion or addition of a sequence of one or more nucleotides, and having the same or similar function as that of the nucleotide sequence set forth in SEQ ID NO: 1 or SEQ ID No. 2.

In some specific embodiments of the present invention, the sequence of more than one nucleotides has 2, 3, 4, 5, 6, 7, 8, 9, 10, 11 12 or 13 nucleotides. As a preference, the more than one nucleotide sequences as described in SEQ ID NO: 1 (Foxo gene) are 2, 3, 4, 5, 6 or 7 nucleotide sequences; the more than one nucleotide sequences set forth in SEQ ID NO: 2 (Ebony gene) are 2, 3, 4, 5, 6, 7, 8, 9, 10, 11 12 or 13 nucleotide sequences.

The present invention further provides a recombinant DNA comprising the polynucleotide (Foxo gene or Ebony gene).

The present invention further provides an expression vector, which is inserted with the recombinant DNA and uses a microorganism, an animal cell or a plant cell as the host cell.

The present invention further provides a transformant transformed with the expression vector

The present invention further provides use of the polynucleotide (Foxo gene or Ebony gene) in identification of a species; wherein the species include the A. m. sinisxinyuan.

Additionally, the present invention further provides use of the polynucleotide (Foxo gene or Ebony gene) in genetic diversity of species resources.

The present invention further provides use of the polynucleotide (Foxo gene or Ebony gene) in cold resistance.

The present invention further provides a primer set for identifying A. m. sinisxinyuan, comprising primers capable of amplifying the said polynucleotide (Foxo gene or Ebony gene).

The present invention further provides a kit for identifying A. m. sinisxinyuan, comprising the primer set.

The present invention further provides a method for identifying A. m. sinisxinyuan, comprising:

step 1: Obtaining the DNA of a species to be tested;

step 2: by means of gene alignment, if the polynucleotide (Foxo gene or Ebony gene) is contained, the species to be tested is A. m. sinisxinyuan; while if the polynucleotide (Foxo gene or Ebony gene) is absent, the species to be tested is not A. m. sinisxinyuan.

The present invention provides a specific gene sequence of A. m. sinisxinyuan. The whole genome sequencing refers to sequencing all genes in the genome of an organism to determine the DNA base sequence thereof. The whole genome sequencing has a wide coverage and can detect all of the genetic information in the genome of an individual with high accuracy. Each individual inherits the DNA genetic information from parents at the beginning of a fertilized egg; the genetic information is carried for the whole life and hardly changes. The whole genome sequencing is a process performed by applying a new generation high-throughput DNA sequencer for individual whole genome sequencing with a coverage rate of 10-20 times, and then comparing with the precise map of the genome of the same species to obtain the complete whole genome sequence of the individual and thus deciphering all the genetic information of the individual. For the whole genome sequenced individual, by means of sequence alignment, a large amount of single nucleotide polymorphism (SNP) sites specific to a particular species (strain) can be found. The present invention for the first time identified the critical genes by which A. m. sinisxinyuan adapted to cold climates. 2 genes are included: Foxo and Ebony. These genes play important roles in the differentiation of A. m. sinisxinyuan from the bees in other regions and in the adaptive evolution to the local environment.

The Foxo gene or Ebony gene provided in the present invention can be used to identify A. m. sinisxinyuan from other species, can also be used for studying the genetic diversity of species resources of bees, and can further be used for studying cold resistance genes. This will fill the gap in the research field of A. m. sinisxinyuan by Chinese researchers.

### DESCRIPTION OF DRAWINGS

The patent or application file contains at least one drawing executed in color. Copies of this patent or patent application publication with color drawing(s) will be provided by the office upon request and payment of the necessary fee.

In order to illustrate the examples of the present invention or the technical solutions in the prior art more clearly, the drawings which are required for use in the examples or the prior art descriptions will be briefly described below.

FIG. 1 shows the gene trees of the 2 genes in A. m. sinisxinyuan and other representative populations (European dark bee and African honey bee).

FIG. 2 shows the graph of genomic DNA extraction results; large fragments of DNA with high-quality were obtained from all the samples, and no significant degradation was shown in any of the samples; the “Standard” in the graph is the standard sample loaded with 5 ul (10 ng/ul); M-1 is the Trans 2k plus DNA molecular weight standard, loaded with 2 ul; M-2 is the Trans 15k plus DNA molecular weight standard, loaded with 2 ul; the rest are the sample DNAs;

FIG. 3(A) shows that several statistics such as F_{ST}, Tajima's D and θ_{π} were used to scan the two genes and apparent selected signals were both detected, indicating that these genes were subjected to a specific natural selection in A. m. sinisxinyuan;

FIG. 3(B) shows that the two genes have special DNA sequences; the SNP sites of the gene region have significant genotype differences as compared to the other subspecies.

### DETAILED DESCRIPTION OF EMBODIMENTS

The present invention discloses a gene of A. m. sinisxinyuan and application thereof, and those skilled in the art can use the content herein for reference to improve the technological parameters appropriately to achieve it. It should be particularly noted that all the similar substitutions and alterations will be apparent to those skilled in the art, and are deemed to be included in the present invention. The method and use of the present invention have been described by way of preferred embodiments, and it will be apparent to the related personnel that the method and use described herein may be altered or appropriately modified and combined to achieve and apply the technology of the present invention without departing from the content, spirit and scope of the present invention.

The method for obtaining and detecting the gene provided in the present invention is as the follows:

1. Sample collection. Collecting living honey bee samples and immediately putting them into 90% ethanol for storage. In further specific embodiments of the present invention, in addition to 90% ethanol for storage, ethanol with higher purities, or liquid nitrogen, dry ice and other low-temperature preservation methods can also be used for sample storage.

2. Extracting high-quality DNA (deoxyribonucleic acid) from the samples.

3. Subjecting the DNA samples to Illumina high-throughput sequencing to obtain the raw data of DNA sequences. Any high-throughput sequencing platforms can be used for DNA high-throughput sequencing; in addition to the Illumina platform described above, SOLiD platform, 454 sequencing and other second- or third-generation sequencing platforms can also be used.

4. Filtering out the low-quality sequences. The rules for filtering include: 1) the number of terminal “N” should be less than or equal to 10% of the sequence length; 2) the number of base with sequencing quality lower than 5 should be no more than 50% of the sequence length.

5. Performing sequence alignment using BWA software by taking the western honey bee (Apis mellifera) genome in the NCBI public database as the reference genome (apiMe14.5), wherein the alignment parameter is “-t -k 32-M-R”. The latest version for the reference genome is apiMe14.5, and the reference genome of updated versions can be employed after their publication.

6. Obtaining the SNP genotypes of a population using the SAMtools' mpileup program, and filtering the obtained genotypes to obtain the final results. The rules for filtering are: 1) the quality value should be no less than 20; 2) SNPs within 5 bases from a sequence gap should be filtered out; 3) the sequencing depth should be greater than or equal to 4, and less than or equal to 1000; the programs for obtaining SNP genotypes can be any programs that is capable of performing variation test, including the above-described SAMtools, and GATK, CLC and other programs; 4) SNP sites with 3 or more genotypes are removed.

All of the raw materials and reagents used in gene of A. m. sinisxinyuan and use thereof provided in the present invention are commercially available.

The present invention is further illustrated in combination with the following examples:

### EXAMPLE 1

(1) Honey bee samples were collected and DNA was extracted; samples with the OD value of the DNA being 1.8-2.0, content over 1.5 μg were considered to be qualified.

(2) A Library was Constructed with the Qualified DNA Samples:

The DNA samples tested to be qualified were broken randomly into fragments with a length of 350 bp via a Covaris crusher. TruSeq Library Construction Kit was employed to construct the library and the reagents and consumables recommended in the manual were used strictly. DNA fragments were subjected to end-repair, tail addition, sequencing adaptor addition, purification, PCR amplification and other steps to accomplish the preparation of the whole library. The well-constructed library was sequenced by illumina HiSeq.

(3) Library Inspection:

After the library was constructed, Qubit2.0 was used first for preliminary quantification and the library was diluted to 1 ng/μl; subsequently, the insert size of the library was detected with Agilent 2100. After the insert size met the expectation, the effective concentration of the library was accurately qualified by Q-PCR method (effective concentration of the library >2 nM) to ensure the quality of the library.

(4) Sequencing on Machine:

With library inspection qualified, illumina HiSeq sequencing was conducted according to the effective concentration of the library and requirements of data output.

(5) Quality Control:

Sequenced Reads or raw reads obtained by sequencing contain low-quality reads with adaptors. In order to ensure the quality of information analysis, raw reads must be filtered to obtain clean reads, and all of the following analyses were based on the clean reads. Data processing steps are as follows:

a. removing paired-end reads with adaptors;

b. such paired-end reads need to be removed when the content of N contained in the single-end sequencing read exceeds 10% of the read length;

c. such paired-end reads need to be removed when the number of low-quality (Q<=5) base contained in the single-end sequencing read exceeds 50% of the read length.

A total of 179 million high-quality double-end sequencing sequences (read length 100 bp) were obtained by re-sequencing 10 A. m. sinisxinyuan individuals, with a total data volume being 17.9 G.

(6) Sequence Alignment:

Sequence (clean reads) alignment was conducted with the BWA software, and default values were adopted for all parameters except “-t-k 32-M-R”. With Amel 4.5 (derived from NCBI) taken as the reference genome, the bam files obtained from alignment were sorted with the SAMtools software and the duplicated sequences were removed.

After sequence alignment, a sequencing depth of 8 × was obtained with a genome coverage rate of about 90%.

(7) SNP Detection:

After the bam files were obtained, SNP detection was performed. SNP (single nucleotide polymorphism) mainly refers to DNA sequence polymorphism caused by a single nucleotide variation on genomic level, including transition, transversion, etc. of a single base. SAMTOOLS (mpileup-m2-F 0.002-d 1000) was used for individual SNP detection. In order to reduce the error rate of SNP detection, the following criteria were selected for filtering:

a. the support number of SNP reads is no less than 4;

b. the quality value (MQ) of SNPs is no less than 20;

A total of 1,409,113 SNP sites were detected in A. m. sinisxinyuan as compared with the reference genome.

(8) SNP Annotation:

ANNOVA is an efficient software tool that uses the latest information to annotate gene variations detected from multiple genomes. ANNOVAR can perform gene-based annotation, region-based annotations, filter-based annotation, and other functionalities as long as the chromosomes where the variation is located, start sites, stop sites, reference nucleotides and variant nucleotides are given. In view of the powerful annotation capability and international acceptance of ANNOVAR, it was used to annotate SNP detection results.

The annotation result shows that among the 1,409,113 SNPs, 28,067 are located in the upstream region of the gene (within 1 Kb), 24,778 are located in the downstream region of the gene (within 1 Kb), 62,289 are located in the exon region, 657,772 are located in the intron region, 110 are located at the cleavage sites, and 633,186 are located in the remaining non-gene regions.

(9) The corresponding gene sequences can be extracted with GATK kit, using the reference genomic sequence and the detected SNP sequences.

The results are as shown in FIG. 1 to FIG. 3.

FIG. 1 shows the gene trees of the 2 genes in A. m. sinisxinyuan and other representative populations (European dark bee and African honey bee).

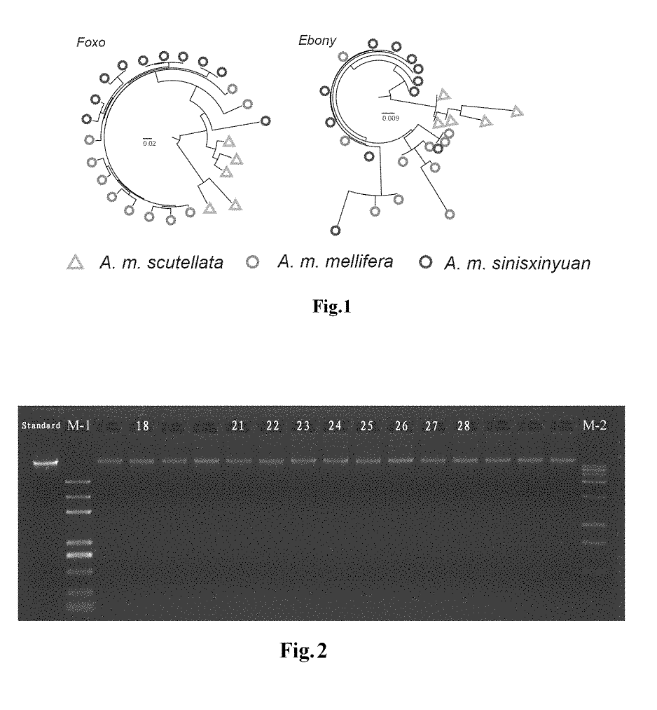

FIG. 2 shows the graph of genomic DNA extraction results; large fragments of DNA with high-quality were obtained from all the samples, and no significant degradation was shown in any of the samples; the “Standard” in the graph is the standard sample loaded with 5 ul (10 ng/ul); M-1 is the Trans 2k plus DNA molecular weight standard, loaded with 2 ul; M-2 is the Trans 15k plus DNA molecular weight standard, loaded with 2 ul; the rest are the sample DNAs;

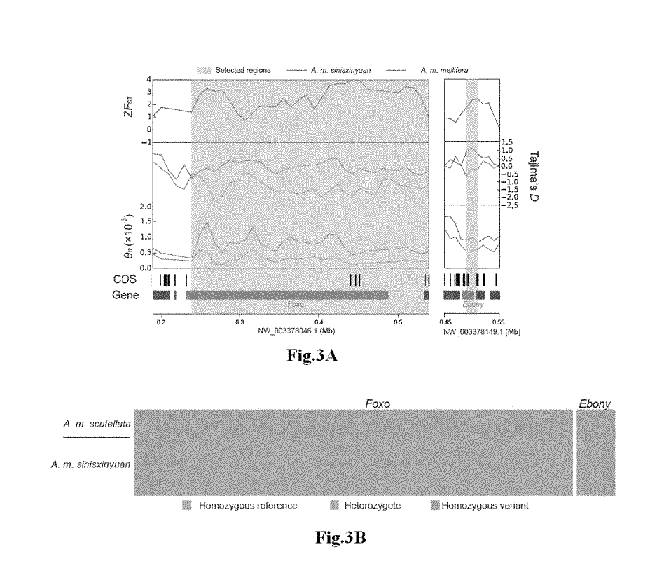

FIG. 3(A) shows that several statistics such as F_{ST}, Tajima's D and θ_{π} were used to scan the two genes and apparent selected signals were both detected, indicating that these genes were subjected to a specific natural selection in A. m. sinisxinyuan; FIG. 3(B) shows that the two genes have special DNA sequences; the SNP sites of the gene region have significant genotype differences as compared to the other subspecies.

The foregoing are only preferred embodiments of the present invention, it should be noted that a number of improvements and modifications may be made thereto by an ordinary skilled in the art without departing from the principles of the present invention, and these improvements and modifications should also be deemed to be within the protection scope of the present invention.

1. A polynucleotide having:
(I) the nucleotide sequence set forth in SEQ ID No. 1 or SEQ ID No. 2; or
(II) a sequence complementary to the nucleotide sequence set forth in SEQ ID No. 1 or SEQ ID No. 2; or
(III) a sequence which encodes the same protein as that the nucleotide sequence of (I) or (II) does but differs from the nucleotide sequence of (I) or (II) due to genetic codon degeneracy; or
(IV) a nucleotide sequence having the nucleotide sequence obtained from the nucleotide sequence set forth in SEQ ID NO: 1 or SEQ ID No. 2 by substitution, deletion or addition of a sequence of one or more nucleotides, and having the same or similar function as that of the nucleotide sequence set forth in SEQ ID NO: 1 or SEQ ID No. 2.

2. The polynucleotide according to claim 1, wherein the sequence of more nucleotides has 2, 3, 4, 5, 6, 7, 8, 9, 10, 11, 12 or 13 nucleotides.

3. A recombinant DNA comprising the polynucleotide according to claim 1.

4. A method for use in molecular marker-assisted breeding of Apis mellifera by using of the polynucleotide according to claim 1.

5. A method for use in cold resistance by using the polynucleotide according to claim 1.

6. A method for identifying A. m. sinisxinyuan, comprising:
step 1: obtaining the DNA of a species to be tested;
step 2: by means of gene alignment, if the polynucleotide according to claim 1 is present, the species to be tested is A. m. sinisxinyuan; while if the polynucleotide according to claim 1 is absent, the species to be tested is not A. m. sinisxinyuan.
